(19) United States
(12) Patent Application Publication
(10) Pub. No.: US 2024/0170143 A1
(43) Pub. Date: May 23, 2024

(54) MEDICAL INFORMATION PROCESSING APPARATUS AND MEDICAL INFORMATION PROCESSING METHOD

(71) Applicant: CANON MEDICAL SYSTEMS CORPORATION, Otawara-shi (JP)

(72) Inventor: Yohei KAMINAGA, Otawara (JP)

(73) Assignee: CANON MEDICAL SYSTEMS CORPORATION, Otawara-shi (JP)

(21) Appl. No.: 18/512,120

(22) Filed: Nov. 17, 2023

(30) Foreign Application Priority Data
Nov. 17, 2022 (JP) 2022-184219

(51) Int. Cl. G16H 40/40

(52) U.S. Cl. CPC G16H 40/40

(57) ABSTRACT

A medical information processing apparatus includes processing circuitry configured to acquire status information indicating a status of a plurality of medical devices connected to each other via a network and/or a status around the plurality of medical devices, generate, based on the status information, operation information indicating an operation to be performed on the medical device, and control a display unit so as to display the generated operation information.

### CROSS REFERENCE TO RELATED APPLICATION

This application is based upon and claims the benefit of priority from the prior Japanese Patent Application No. 2022-184219, filed on Nov. 17, 2022, the entire contents of which are incorporated herein by reference.

### FIELD

Embodiments disclosed in the present specification and drawings relate to a medical information processing apparatus and a medical information processing method.

### BACKGROUND

In the case of performing an inspection using a medical device such as an X-ray computed tomography (CT) device, semi-automation has been advanced in order to enhance inspection efficiency and perform inspection with high reproducibility. In addition, remote operation of the medical device via a communication network is also in progress.

With such a change in an inspection method, it is considered that a user (an operator) of the medical device, such as a radiographer, remotely operates a plurality of medical devices alone. However, it is significantly difficult for one user to perform the inspection of a plurality of medical devices in parallel.

### BRIEF DESCRIPTION OF THE DRAWINGS

FIG. 1 is a diagram illustrating an example of a configuration of a medical information processing apparatus according to an embodiment;

FIG. 2 is a block diagram illustrating an example of a configuration of a server according to the embodiment;

FIG. 3 is a block diagram illustrating an example of a medical operations execution plan generation function according to the embodiment;

FIG. 4 is a block diagram illustrating an example of a configuration of a terminal according to the embodiment;

FIG. 5 is a diagram illustrating an example of a sequence between the server and the terminal according to the embodiment;

FIG. 6 is a diagram illustrating an example of a plurality of workflows allocated to a plurality of medical devices;

FIG. 7 is a flowchart showing an example of processing of generating a medical operations execution plan according to the embodiment;

FIG. 8 is a flowchart showing an example of processing of specifying a transitionable timing according to the embodiment;

FIG. 9 is a diagram illustrating an example of a plurality of workflows in the medical operations execution plan according to the embodiment;

FIG. 10 is a diagram illustrating a first example of a screen displayed on a display of the terminal according to the embodiment;

FIG. 11 is an enlarged view of a part of a timeline of the screen example in FIG. 10;

FIG. 12A is an enlarged view of an execution status of the screen example in FIG. 10;

FIG. 12B is a diagram illustrating another screen example illustrating an execution status; and

FIG. 13 is a diagram illustrating a second example of the screen displayed on the display of the terminal according to the embodiment.

### DETAILED DESCRIPTION

Hereinafter, an embodiment of a medical information processing apparatus will be described with reference to the drawings. The embodiment described below is configured as a system including a plurality of devices, i.e., a server and a terminal, but the embodiment is not limited to this. That is, the medical information processing apparatus according to the embodiment may be configured as a single device that includes the functions of the server in the terminal. It is noted that, in the following description, components having substantially the same functions and configurations are denoted by the same reference numerals, and redundant description will be made only when necessary.

A medical information processing apparatus 1 according to an embodiment is configured to be able to collectively manage a plurality of medical devices connected via a communication network. In addition, as described later, the medical information processing apparatus 1 is configured to automatically select a medical device to be operated by a user (an operator) and a medical process (hereinafter, also simply referred to as “process”) to be executed by the medical device, and to present the medical device and the process to the user.

The medical information processing apparatus 1 is configured to acquire status information indicating a status of a plurality of medical devices and/or a status around the plurality of medical devices and to present, to the user (operator), operation information generated based on the status information. The operation information indicates an operation to be performed on the medical device. Here, the “operation” that the user performs on the medical device may include not only (direct) operations on the medical device but also operations performed via the medical device. As an example of the latter, the user may instruct (operate) a local staff working in a room with a medical device by having a display on the medical device show an operation to be performed by the local staff (e.g., moving a patient).

In addition to the status information, the medical information processing apparatus 1 may also acquire information being fed back or notified to the user from the local staff and/or an operating instruction from the local staff. In this case, the operation information may be generated based on the acquired information and/or the operating instruction.

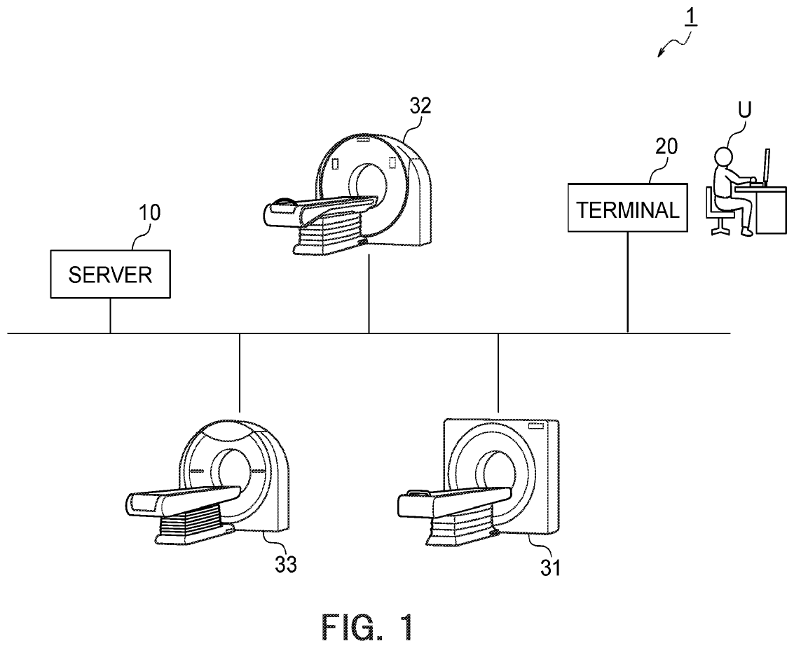

As illustrated in FIG. 1, the medical information processing apparatus 1 includes a server 10, a terminal 20, and a plurality of medical devices 31, 32, and 33. These elements are communicably connected to each other via a communication network such as in-hospital LAN or the Internet. For example, the terminal 20 may be connected to medical devices disposed in a plurality of hospitals via the Internet or the like. It is noted that, in the following description, the medical devices 31, 32, and 33 are also referred to as medical devices A, B, and C, respectively.

The server 10 generates a medical operations execution plan based on various types of information to be described later. The medical operations execution plan is a plan for executing a plurality of workflows related to medical operations in parallel using the plurality of medical devices 31, 32, and 33.

The terminal 20 is a terminal operated by a user U (a radiographer or the like) who remotely operates the plurality of medical devices 31, 32, and 33. The terminal 20 is disposed in an operation room for operating the plurality of medical devices 31, 32, and 33. For example, the terminal 20 is disposed in a centralized support center existing in a place remote from the medical devices 31, 32, and 33. It is noted that the plurality of medical devices 31, 32, and 33 may be disposed in a plurality of rooms (inspection rooms) in one hospital respectively, or may be disposed in a plurality of hospitals respectively.

The medical devices 31, 32, and 33 are X-ray computed tomography devices (CT devices) in the present embodiment. It is noted that the medical device may be another inspection device such as a magnetic resonance imaging device (an MRI device), an X-ray diagnostic device, a positron emission tomography (PET) device, or an ultrasonic diagnostic device. In addition, the medical device is not limited to the inspection device, and may be a device for treatment (such as a surgical robot).

Although not illustrated herein, the medical information processing apparatus 1 may include an electronic medical record system, a reconstruction server that reconstructs a diagnostic image from imaging data, a picture archiving and communication system (PACS; a medical image management system), and the like. In addition, the medical information processing apparatus 1 may include a camera of a room in which the medical device is installed, a shared monitor of an inspection waiting room, and the like.

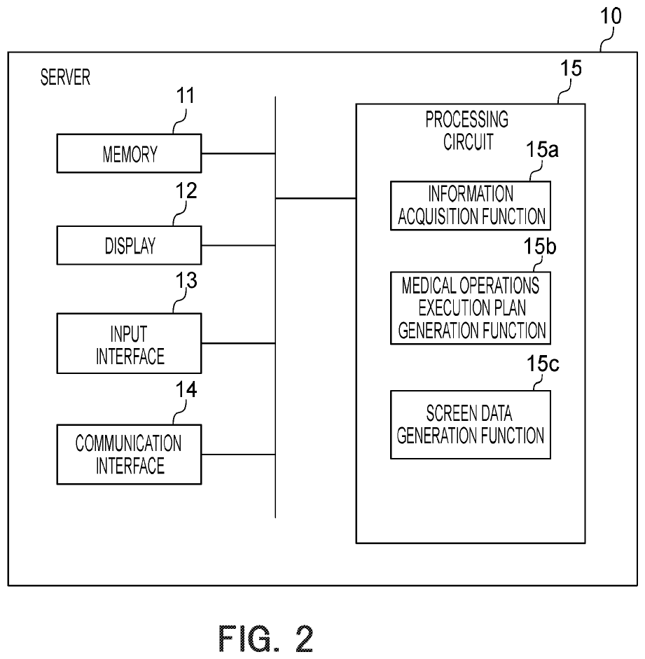

As illustrated in FIG. 2, the server 10 includes a memory 11, a display 12, an input interface 13, a communication interface 14, and a processing circuit 15. Hereinafter, details of each configuration of the server 10 will be described.

The memory 11 is connected to the processing circuit 15 and stores various types of information (for example, CT image data acquired from the reconstruction server or the PACS) used in the processing circuit 15. The memory 11 is implemented by, for example, a random access memory (RAM), a semiconductor memory element such as a flash memory, a hard disk, an optical disk, or the like.

Furthermore, the memory 11 stores various programs necessary for the processing circuit 15 to execute each function, various data processed by the programs, and the like. It is noted that the various types of data handled in the present specification are typically digital data.

The display 12 includes, for example, a liquid crystal display, a cathode ray tube (CRT) display, or the like. The input interface 13 receives various input operations, converts the received input operations into electrical signals, and outputs the electrical signals to the processing circuit 15. The input interface 13 is implemented by, for example, a mouse, a keyboard, a touch panel, a button, a trackball, a manual switch, a foot switch, a joystick, or the like. It is noted that, when the server 10 and the terminal 20 are integrated with each other, the display 12 and the input interface 13 function as the display 22 and the input interface 23 of the terminal 20.

The communication interface 14 implements various communication protocols according to a form of an in-hospital network. The communication interface 14 implements communication with other systems and devices via the in-hospital network according to the various communication protocols. In the present embodiment, the server 10 is connected to the in-hospital network via the communication interface 14, and communicates with the terminal 20 and the medical devices 31, 32, and 33. Furthermore, the server 10 communicates with other systems and devices such as the electronic medical record system and the PACS as necessary.

The processing circuit 15 is a calculation circuit that performs various calculations and controls the operation of the server 10. The processing circuit 15 generates the medical operations execution plan based on the workflow allocated to the medical devices 31, 32, and 33. In addition, the processing circuit 15 generates information to be displayed on the terminal 20 based on information (a CT image and the like) acquired from the medical devices 31, 32, and 33, the reconstruction server, and the like, and transmits the generated information to the terminal 20.

In order to implement such a function, as illustrated in FIG. 2, the processing circuit 15 has an information acquisition function 15a, a medical operations execution plan generation function 15b, and a screen data generation function 15c. The information acquisition function 15a is an example of an information acquisition unit, a status information acquisition unit or a workflow information acquisition unit. The medical operations execution plan generation function 15b is an example of a medical operations execution plan generation unit.

In the present embodiment, each processing function executed by a corresponding one of the information acquisition function 15a, the medical operations execution plan generation function 15b, and the screen data generation function 15c is stored in the memory 11 in the form of a program executable by a computer. The processing circuit 15 is a processor, and implements a function corresponding to each program by reading and executing the program from the memory 11. In other words, the processing circuit 15 in a state of reading each program has each function indicated in the processing circuit 15 of FIG. 2.

It is noted that FIG. 2 illustrates a case in which each processing function of the information acquisition function 15a, the medical operations execution plan generation function 15b, and the screen data generation function 15c is implemented by the single processing circuit 15, but the embodiment is not limited thereto. For example, the processing circuit 15 may be configured by combining a plurality of independent processors, and each processor may implement each processing function by executing each program. Furthermore, each processing function of the processing circuit 15 may be implemented by being appropriately distributed to or integrated into a single or a plurality of processing circuits.

Next, details of each processing function will be described.

The information acquisition function 15a acquires various types of information. For example, information on the workflow allocated to the medical devices 31, 32, and 33 is acquired from the electronic medical record system or the like. In addition, the information acquisition function 15a acquires various types of information (device screen information and the like) from the medical devices 31, 32, and 33 and the PACS. Furthermore, the information acquisition function 15a may acquire information photographed by a camera provided in a room (an inspection room, a diagnostic room, a treatment room, an operating room for surgery or the like) in which the medical device is installed, biological information (pulse, body temperature, and the like) obtained from an inspection device attached to a patient, and specimen inspection result information such as a blood gas inspection, central inspection, and inspection of a respiratory rate.

The information acquisition function 15a acquires status information indicating a status of the medical devices 31, 32 and 33 and/or a status around the medical devices 31, 32, 33. The information acquisition function 15a functions as a status information acquisition unit. The information indicating status of the medical devices 31, 32 and 33 is, for example, information indicating an operating status or an operating phase (e.g., whether an inspection has been completed or not) of the medical devices 31, 32 and 33. The information indicating status around the medical devices 31, 32 and 33 is, for example, an image showing state of a room where the medical device 31, 32 or 33 is installed, the image being captured by a camera installed in the room.

The information acquisition function 15a also acquires workflow information indicating a plurality of medical processes executed in parallel on the plurality of medical devices 31, 32 and 33. The workflow information may be obtained from the medical operations execution plan. In this case, the operation information may be generated based on the workflow information and the status information.

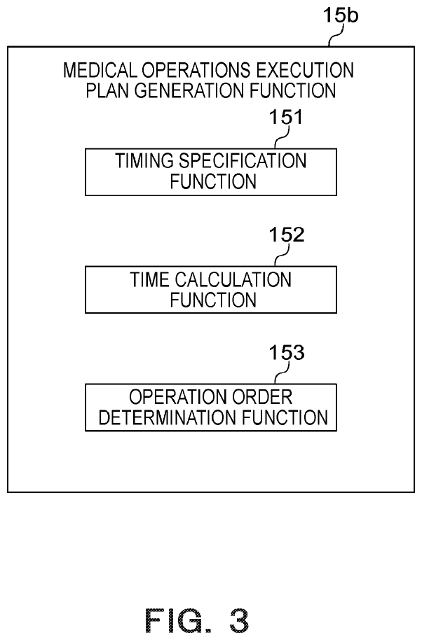

The medical operations execution plan generation function 15b generates a medical operations execution plan for executing a plurality of workflows in parallel using the plurality of medical devices 31, 32, and 33. As illustrated in FIG. 3, the medical operations execution plan generation function 15b has a timing specification function 151 that specifies a timing at which transition can be performed by a user between workflows, a time calculation function 152 that calculates an execution time or the like of each process constituting the workflow, and an operation order determination function 153 that determines an order in which the user operates the plurality of medical devices.

The timing specification function 151 specifies, for each of the plurality of workflows, a timing at which a process of a certain medical device can transition to a process of another medical device. For example, in a case where the medical device is a CT device, the timing specification function 151 specifies, as a transitionable timing, each of a timing before main scanning is performed, a timing at which main scanning is in progress, and a timing after post-processing is completed. Details of a timing specification method will be separately described.

The time calculation function 152 calculates a process execution time of each process included in the plurality of workflows and a grace time between processes. The process execution time is a time required for executing the process. The grace time between the processes is a time allowed until the user returns to a work of a first medical device after leaving the work of the first medical device in a case where a process of the first medical device transitions (moves) to a process of a second medical device.

For example, the time calculation function 152 calculates the process execution time and the grace time based on at least one of performance information of the medical devices 31, 32, and 33, patient individual information, characteristic information of the user, and a protocol. In a case where the medical devices 31, 32, and 33 are CT devices, the performance information of the medical devices includes an imaging time, a reconstruction time, and the like for each plan, and the patient individual information is a past inspection or a treatment record of a patient and includes an imaging time in the past inspection, a time required for post-processing, and the like. The characteristic information of the user is characteristic information of a technician or the like who operates the medical device, and includes information such as a proficiency level, an operation speed, a preference, and the like with respect to the medical device. The proficiency level may be, for example, information evaluated for each inspection, each portion, and each device. The operation speed may be learned from past operation results. The user preference is information such as a preference regarding a transition speed (fast/normal/slow) of the process. It is noted that the patient individual information and the user characteristic information may be acquired from the server (the PACS or the like). The protocol is, for example, an imaging protocol of the CT device. The imaging protocol may be estimated from inspection contents by the medical device.

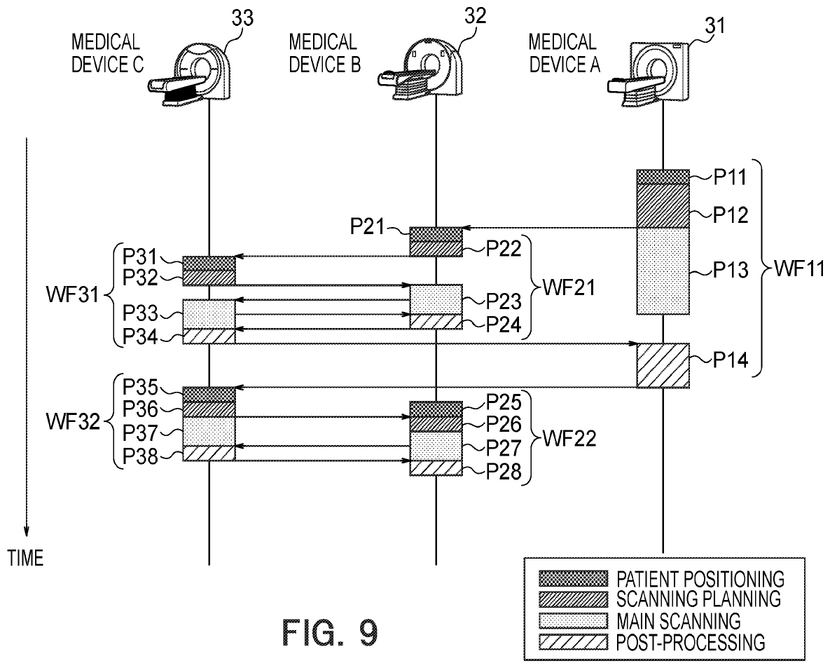

The operation order determination function 153 determines an operation order for the user to sequentially operate the plurality of medical devices 31, 32, and 33 based on the calculated process execution time and grace time, and the specified timing. According to the determined operation order, when the user sequentially operates the plurality of medical devices 31, 32, and 33, a plurality of workflows can be executed in parallel by the plurality of medical devices. Details of an operation order determination method will be separately described with reference to FIG. 9.

The screen data generation function 15c generates screen data (display information) for displaying, on the display of the terminal 20, the information acquired by the information acquisition function 15a and the information generated by the processing circuit 15 such as the medical operations execution plan. The generated screen data is transmitted to the terminal 20 via the communication interface 14.

The screen data generating function 15c generates operation information indicating operations to be performed on the medical devices 31, 32 and 33 based on the status information acquired by the information acquiring function 15a. That is to say, the screen data generating function 15c functions as an operation information generating unit. The screen data generating function 15c also generates screen data for displaying the generated operation information on the display of the terminal 20. For example, the screen data generating function 15c generates, based on the status information, information included in a timeline TL, a side view SV, an operation view OV, an execution status ES and a process flow PF described below as the operation information.

The screen data generation function 15c may generate, when there are a plurality of the operations to be performed on the plurality of medical devices 31, 32 and 33, a plurality of pieces of the operation information based on the status information. Each of the pieces of the operation information is generated for a corresponding one of the operations. In this case, the display control function 25b of the terminal 20 may be configured to control a display 22 so as to display the plurality of pieces of operation information side by side on a time axis. The side view SV described later may be displayed in this manner. The display control function 25b may also be configured to control the display 22 so as to display the plurality of pieces of operation information side by side in a timeline. The timeline TL described later may be displayed in this manner.

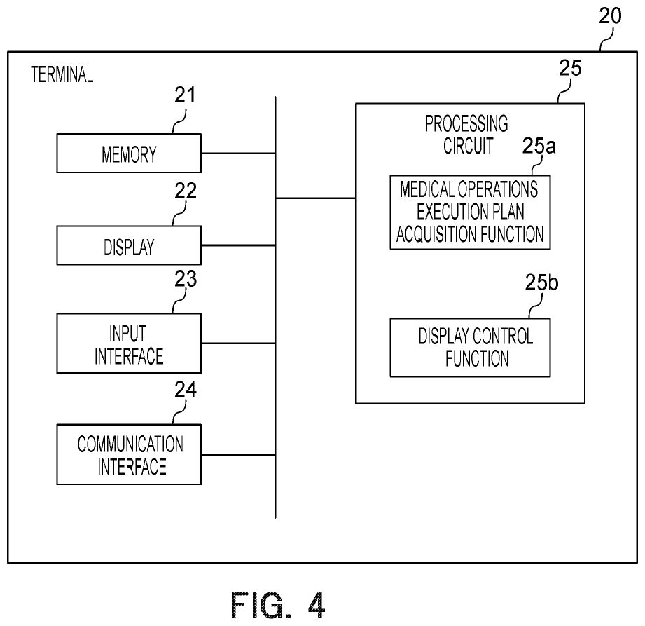

As illustrated in FIG. 4, the terminal 20 includes a memory 21, a display 22, an input interface 23, a communication interface 24, and a processing circuit 25. The memory 21 is connected to the processing circuit 25 and stores various types of information used in the processing circuit 25. The memory 21 is implemented by, for example, a random access memory (RAM), a semiconductor memory element such as a flash memory, a hard disk, an optical disk, or the like.

The display 22 displays timelines of a plurality of workflows, device information on the medical devices, and the like based on the screen data received from the server 10. In the present embodiment, the display 12 is formed of, for example, a liquid crystal display, a cathode ray tube (CRT) display, or the like.

The input interface 23 receives various input operations from the user, converts the received input operations into electrical signals, and outputs the electrical signals to the processing circuit 25. The input interface 23 is implemented by, for example, a mouse, a keyboard, a touch panel, a trackball, a manual switch, a foot switch, a button, a joystick, or the like.

The communication interface 24 implements various communication protocols according to the form of the in-hospital network. The communication interface 24 implements communication with another device such as the server 10 via the in-hospital network according to various communication protocols.

The processing circuit 25 is a calculation circuit that performs various calculations, and includes a medical operations execution plan acquisition function 25a that acquires the medical operations execution plan from the server 10, and a display control function 25b that controls display contents of the display 22. The medical operations execution plan acquisition function 25a is an example of a medical operations execution plan acquisition unit, and the display control function 25b is an example of a display control unit.

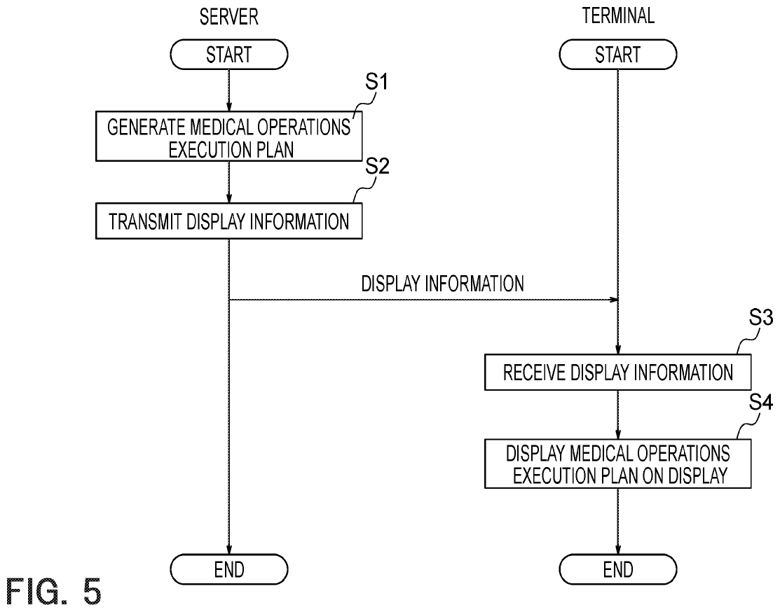

Next, an example of a sequence between the server 10 and the terminal 20 will be described with reference to FIG. 5. On the server 10 side, the medical operations execution plan generation function 15b generates a medical operations execution plan (step S1), and the screen data generation function 15c generates screen data (display information) of the medical operations execution plan and transmits the screen data to the terminal 20 via the communication interface 14 (step S2). On the terminal 20 side, the medical operations execution plan acquisition function 25a receives the display information via the communication interface 24 (step S3), and the display control function 25b displays the medical operations execution plan on the display 22 based on the received display information (step S4).

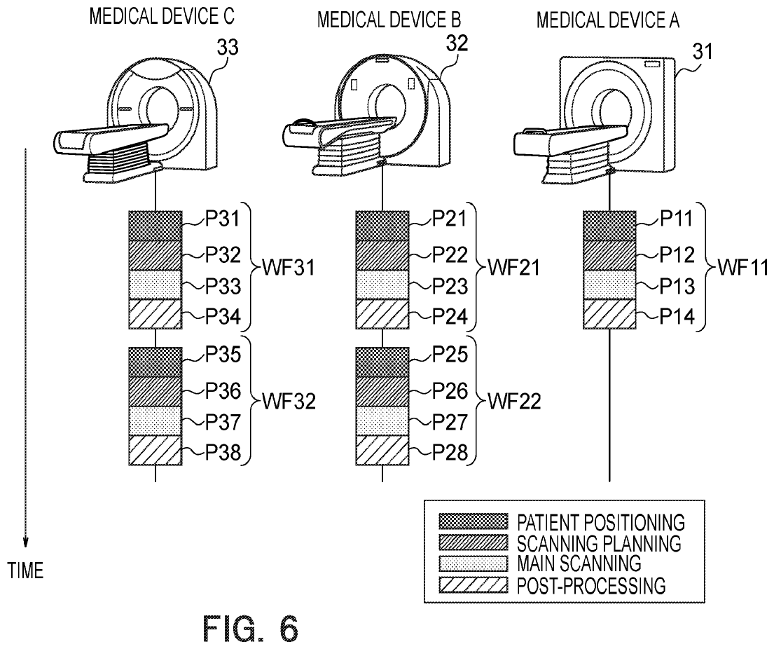

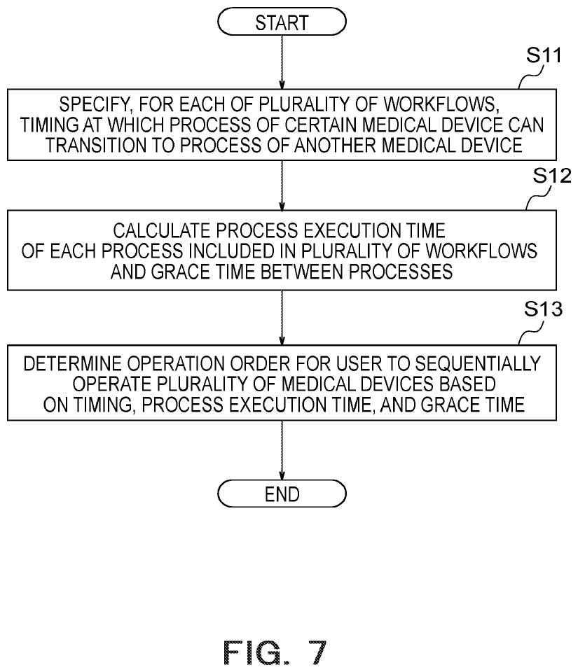

Next, an example of step S1 of generating the medical operations execution plan according to the present embodiment will be described with reference to FIGS. 6 to 9. In the present example, as illustrated in FIG. 6, it is assumed that a workflow WF11 is allocated to the medical device 31, workflows WF21 and WF22 are allocated to the medical device 32, and workflows WF31 and WF32 are allocated to the medical device 33. The workflow WF11 includes processes P11, P12, P13, and P14. The process P11 is patient positioning (patient care), the process P12 is scanning planning (optimization of imaging conditions), the process P13 is main scanning (imaging and reconstruction), and the process P14 is post-processing (image analysis, processing, transfer, and the like).

The workflow WF21 includes processes P21, P22, P23, and P24, and the workflow WF22 includes processes P25, P26, P27, and P28. The processes P21 and P25 are patient positioning, the processes P22 and P26 are scanning planning, the processes P23 and P27 are main scanning, and the processes P24 and P28 are post-processing.

Similarly, the workflow WF31 includes processes P31, P32, P33, and P34, and the workflow WF32 includes processes P35, P36, P37, and P38. The processes P31 and P35 are patient positioning, the processes P32 and P36 are scanning planning, the processes P33 and P37 are main scanning, and the processes P34 and P38 are post-processing.

It is noted that the workflows WF11, WF21, WF22, WF31, and WF32 may be workflows having different contents.

The timing specification function 151 specifies, for each of the plurality of workflows, a timing at which a process of a certain medical device can transition to a process of another medical device (step S11). For example, when the medical device is a CT device, the timing specification function 151 specifies, as a transitionable timing, each of a timing between scanning planning and main scanning, a timing at which execution of the main scanning is in progress, and a timing after post-processing is completed.

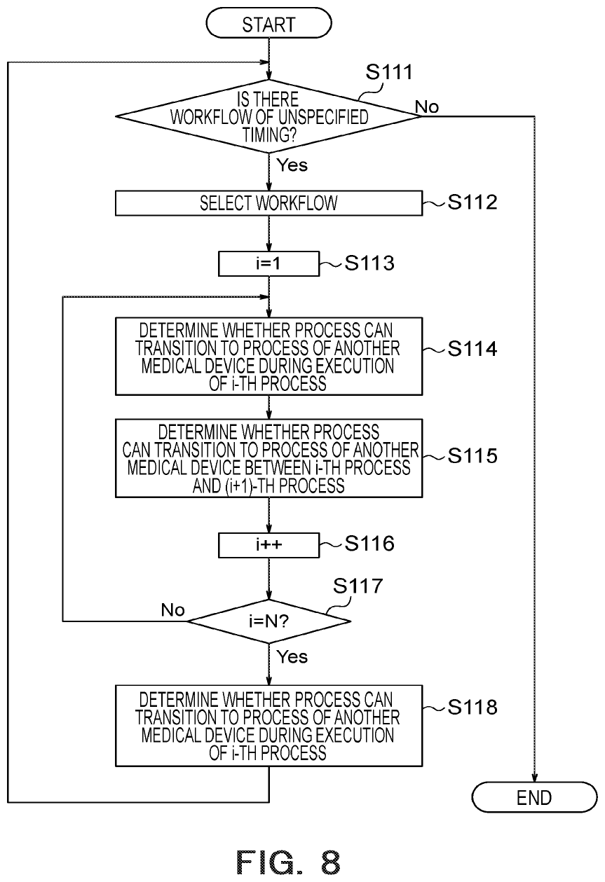

An example of a detailed processing flow in step S11 will be described with reference to FIG. 8.

The timing specification function 151 determines whether there is a workflow for which the transitionable timing has not been specified yet (step S111). When there is an unspecified workflow (S111: Yes), one unspecified workflow is selected (step S112). For example, in a case where a plurality of workflows are allocated to each of the medical devices A, B, and C, as illustrated in FIG. 6, the workflows are selected in the order of the workflows WF11, WF21, WF22, WF31, and WF32. On the other hand, when there is no unspecified workflow (S111: No), the timing specification processing ends.

In step S113, the timing specification function 151 initializes a value of an index. Here, a value of an index i is set to 1.

The timing specification function 151 determines whether transition to a process of a workflow related to another medical device can be performed during execution of the i-th process of the workflow selected in step S112 (step S114). For example, when the i-th process is main scanning, since the user's hand is available until the i-th process ends, the timing specification function 151 determines, as the transitionable timing, a period during which the i-th process is executed.

The timing specification function 151 determines whether transition to the process of another medical device can be performed between the i-th process and the (i+1)-th process of the workflow selected in step S112 (step S115). For example, when the i-th process is a post-processing process or the (i+1)-th process is a main scanning process, the timing specification function 151 determines, as the transitionable timing, a timing after the end of the i-th process. It is noted that the execution order of step S114 and step S115 may be reversed. After step S115 ends, the value of the index i is increased by one (step S116), and it is determined whether the value of the index i is equal to N (step S117). Here, N is the number of processes included in the workflow. When the value of the index i is equal to N (S117: Yes), the process proceeds to step S118. Conversely, when the value of the index i is not equal to N (S117: No), the process returns to step S114.

In step S118, the timing specification function 151 determines whether transition to the process of the workflow related to another medical device can be performed during execution of an N-th process (that is, a final process) of the workflow selected in step S112. This step has the same processing contents as those of step S114. After step S118, the process returns to step S111.

Through the above processing flow in FIG. 8, the transitionable timings with respect to all the workflows allocated to the medical devices 31, 32, and 33 are specified. For example, in the case of the workflow WF11, a timing between the process P12 and the process P13, a timing at which execution of the process P13 is in progress, and a timing after the process P14 is executed are specified as the transitionable timings. In the case of the workflow WF21, a timing between the process P22 and the process P23, a timing at which execution of the process P23 is in progress, and a timing after the process P24 is executed are specified as the transitionable timings, and in the case of the workflow WF22, a timing between the process P26 and the process P27, a timing at which execution of the process P27 is in progress, and a timing after the process P28 is performed are specified as the transitionable timings. In the case of the workflow WF31, a timing between the process P32 and the process P33, a timing at which execution of the process P33 is in progress, and a timing after the process P34 is executed are specified as the transitionable timings, and in the case of the workflow WF32, a timing between the process P36 and the process P37, a timing at which execution of the process P37 is in progress, and a timing after the process P38 is performed are specified as the transitionable timings.

The time calculation function 152 calculates the process execution time of each process included in the plurality of workflows and the grace time between processes (step S12). Specifically, the time calculation function 152 calculates the time required for executing the processes P11 to P14, the processes P21 to P28, and the processes P31 to P38 included in the workflows WF11, WF21, WF22, WF31, and WF32, respectively. For example, the process execution time of the process P11 (patient positioning) of the workflow WF11 is calculated based on the patient individual information (such as the time required for positioning in the past inspection), the process execution time of the process P12 (scanning planning) is calculated based on the imaging protocol and the characteristic information of the user (the proficiency level, the operation speed, and the like), the process execution time of the process P13 (main scanning) is calculated based on the performance information of the medical device 31 (the imaging time, the reconstruction time, and the like), and the process execution time of the process P14 (post-processing) is calculated based on the characteristic information of the user (such as the proficiency level).

In addition, the time calculation function 152 calculates the grace time for each transitionable timing specified in step S11. For example, when a timing between the process P12 and the process P13 of the workflow WF11 is specified as the transitionable timing, the time calculation function 152 calculates the grace time based on the time (that is, the execution time of the process P13) from the end of the process P12 to the end of the process P13. In this case, a time obtained by adding a predetermined time to the executable time of the process P13 may be set to the grace time. For example, the grace time may be determined in consideration of the past operation speed of the user in the same type of medical operations.

The operation order determination function 153 determines an operation order for the user to sequentially operate the plurality of medical devices 31, 32, and 33 based on the timing, the process execution time, and the grace time (step S13). For example, the operation order determination function 153 determines the operation order in consideration of the transitionable timing specified in step S11, and the execution time of each process and the grace time at the transitionable timing calculated in step S12. The operation order is desirably determined so that the operation rates of the medical devices 31, 32, and 33 are as high as possible. For example, the operation order is determined such that the standby time of the user is shortened such that the parallel operation time of the medical devices 31, 32, and 33 is lengthened. It is noted that it is not necessary to set the operation order to transition at all timings specified by the timing specification function 151, and the timing may be selected depending on the purpose such as the operation rate. In this way, the medical operations execution plan in which the execution order of each process is optimized is generated.

It is noted that the operation order determination function 153 may determine the operation order for the user to sequentially operate the plurality of medical devices 31, 32, and 33 only based on the timing without using the process execution time and the grace time, and generate the medical operations execution plan. In this case, for example, the execution time of the same type of process is assumed to be the same between the workflows, and the grace time is assumed to be a constant time corresponding to the type of the next process.

FIG. 9 illustrates an example of the plurality of workflows in the medical operations execution plan generated in step S13. In FIG. 9, the length of each process is a length obtained by reflecting the process execution time. In addition, an arrow indicates a transition timing of a process taken care of by a user such as a technician. As can be seen from FIG. 9, according to the generated medical operations execution plan, while the user sequentially operates the plurality of medical devices 31, 32, and 33, the plurality of medical devices 31, 32, and 33 can execute the plurality of workflows WF11, WF21, WF22, WF31, and WF32 in parallel.

As described above, the medical operations execution plan generation function 15b of the server 10 generates the medical operations execution plan based on a timing specified for each of the plurality of workflows, in which a process of a certain medical device can transition to a process of another medical device at the timing. That is, by using the transitionable timing specified in each workflow, it is possible to generate the medical operations execution plan for executing the plurality of workflows in parallel using the plurality of medical devices.

Furthermore, in the present embodiment, it is possible to generate a more efficient medical operations execution plan by calculating the process execution time of each process in the plurality of workflows and the grace time between processes and considering these times.

Furthermore, in the present embodiment, the process execution time and the grace time can be calculated more accurately by using at least one of the performance information of the medical device, the patient individual information, the characteristic information of the user, and the protocol.

It is noted that the medical operations execution plan generation function 15b may re-generate the medical operations execution plan as necessary. For example, the medical operations execution plan generation function 15b may re-generate the medical operations execution plan in a case where the user executes an unscheduled process that is not included in the medical operations execution plan, in a case where the process of the medical operations execution plan is completed later or earlier than scheduled, or in a case where the condition of a patient suddenly changes. In addition, the medical operations execution plan generation function 15b may re-generate the medical operations execution plan in a case where an abnormal finding is found by an inspection using the medical device and an additional inspection (an additional order) is approved by a doctor.

Furthermore, the medical operations execution plan generation function 15b may re-generate the medical operations execution plan at all times or every time a certain period of time elapses while the medical operations execution plan is being executed.

In a case where the medical operations execution plan is re-generated as described above, the medical operations execution plan may be generated after excluding a process or a workflow that has already been completed. In addition, when a delay in the process is detected, the medical operations execution plan may be re-generated. For example, a longer grace time may be set, or the medical operations execution plan may be changed to prioritize other processes. It is noted that the delay in the process is detected from, for example, a difference between a current process progress status grasped from a camera or the like provided in a room in which the medical device is installed and an initial schedule grasped from an inspection template or the like. In this manner, it is possible to correct a trajectory by reviewing the medical operations execution plan and to suppress or prevent the medical operations execution plan from deviating from the actual progress of the medical operations.

In addition, the medical operations execution plan generation function 15b may generate the medical operations execution plan so as to prevent a process having a high risk of interruption of the inspection or the treatment from being continuously performed. For example, the medical operations execution plan generation function 15b may generate the medical operations execution plan so as to prevent main scanning of the plurality of medical devices from being continuously performed.

<Display Screen of Terminal 20>

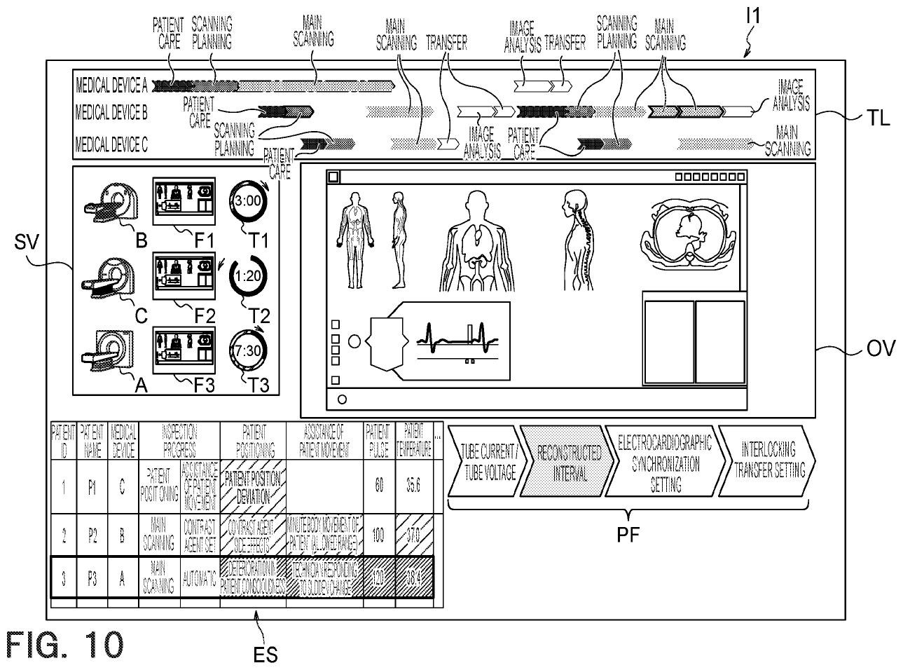

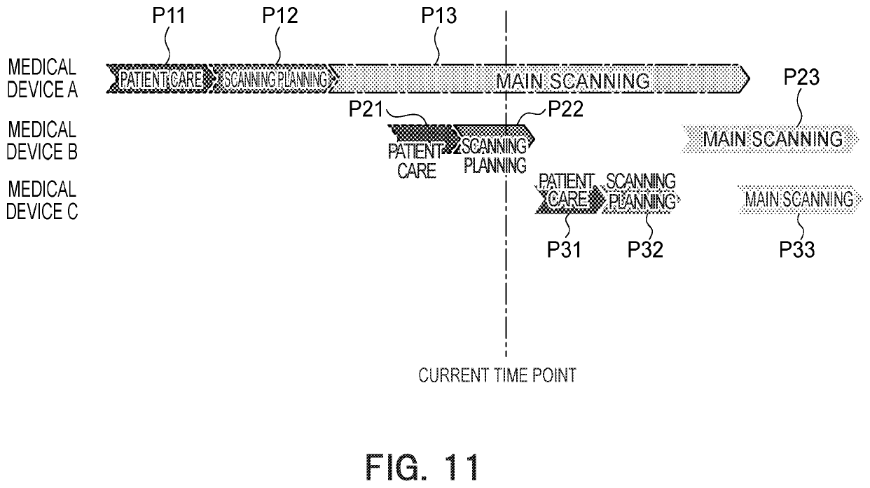

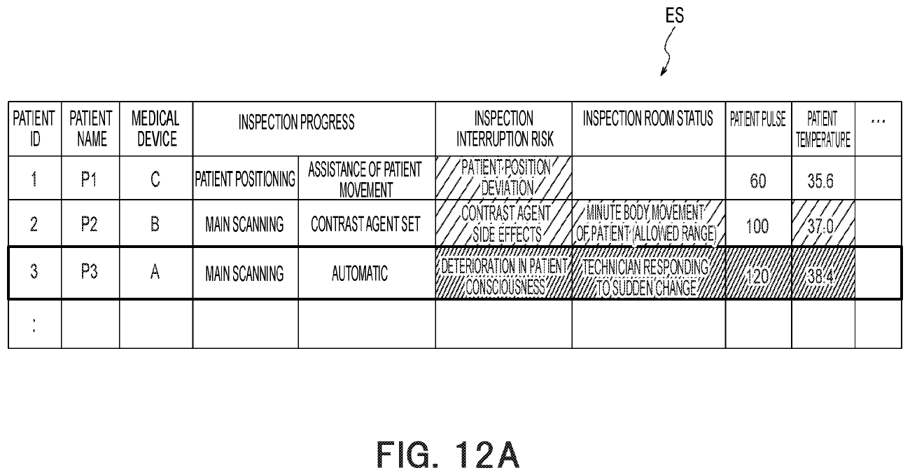

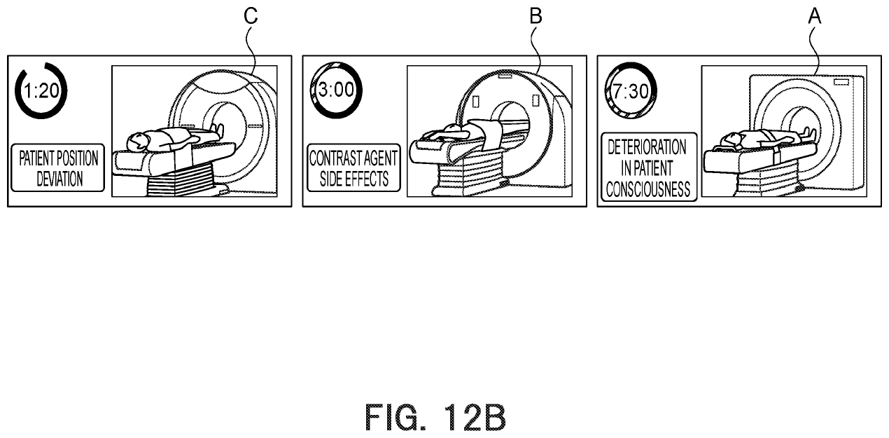

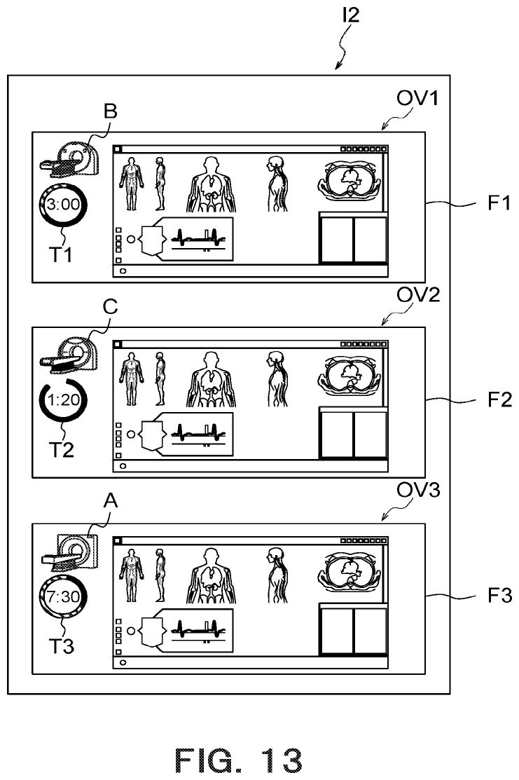

A description will be given as to an example of a screen displayed on the display of the terminal 20 based on the generated medical operations execution plan with reference to FIGS. 10 to 14. FIG. 10 illustrates a first example of the screen displayed on the display of the terminal 20. FIG. 11 illustrates an enlarged view of a part of a timeline of the screen example in FIG. 10, FIG. 12A illustrates an enlarged view of the execution status of the screen example in FIG. 10, and FIG. 12B illustrates another screen example indicating the execution status. FIG. 13 illustrates a second example of the screen displayed on the display of the terminal 20.

As illustrated in FIG. 10, the display control function 25b of the terminal 20 displays a screen 11 on the display 22. The screen 11 includes a timeline TL, a side view SV, an operation view OV, an execution status ES, and a process flow PF. The timeline TL, the side view SV, the operation view OV, the execution status ES, and the process flow PF include the operation information generated by the screen data generation function 15c.

In the timeline TL, a plurality of workflows executed in parallel by the medical devices A, B, and C are collectively displayed in one timeline. Specifically, as illustrated in FIG. 11, processes constituting each workflow are displayed. The timeline TL is displayed based on the medical operations execution plan received from the server 10. In the timeline TL, an overview of the workflow by the plurality of medical devices A, B, and C, and current and future processes are visualized.

Each process displayed in the timeline TL may be displayed in a different display mode depending on the type (kind) of the process. For example, a color in an arrow indicating the process may be changed depending on the type of process, such as a blue color for patient care, a red color for scanning planning, an orange color for main scanning, and a green color for post-processing. In FIGS. 10 and 11, the process type is distinguished by color density in the arrow indicating the process.

Each process displayed in the timeline TL may be displayed in a different display mode depending on the status of the process. The status of the process includes wait for execution, execution in progress, completion, and the like. The process being executed includes a process (such as scanning planning) in which the user needs to operate the medical device and a process (such as main scanning) in which the user does not need to operate the medical device. For example, the color of an edge (a frame) of the arrow indicating the process may be changed depending on the status of the process. In FIGS. 10 and 11, a dotted line indicates a completed process, a solid line indicates a process being executed in which the user needs to operate the medical device, a dashed-dotted line indicates a process being executed in which the user does not need to operate the medical device, and no edge line indicates a process in which the user waits for execution of the medical device. In this way, the timeline TL may display information for identifying the medical process being executed.

When there is an unscheduled process in the processes displayed in the timeline TL, the process may be displayed in a mode different from other processes. For example, the inside of an arrow indicating the unscheduled process is displayed in a color (a purple color or the like) different from the other colors. Furthermore, a frame (a red color or the like) of the color different from the other colors may be provided. In addition, when there is a related process added with the unscheduled process, the process may be displayed in a mode different from other processes.

It is noted that information indicating the current time point may be displayed in the timeline TL as indicated by a vertical line in FIG. 11. In that case, the vertical line moves as the medical operations execution plan progresses. Alternatively, the vertical line may not move and the arrow indicating the process may move. In addition, instead of such vertical line or together with the vertical line, the currently operated process (the process of the medical device which is currently being operated by the user) may be displayed in a mode different from other processes. For example, the currently operated process may be displayed in a specific color, or may be distinguished from other processes by an effect (blinking display or the like) of the process.

In this manner, the display of the timeline TL (such as the color of the arrow of the process) is updated in real time according to the progress of the medical operations execution plan. By viewing the timeline TL, the user can easily grasp what kind of workflow (the process) is scheduled in which medical device, the workflow of the plurality of medical devices, and the execution order of the processes.

In the side view SV, the plurality of medical devices are displayed in the operation order. The side view SV is displayed based on the medical operations execution plan received from the server 10. In FIG. 10, the medical devices are displayed from top to bottom in the operation order. That is, a medical device displayed at the top is a medical device which is currently being operated, and medical devices displayed below the medical device is medical devices scheduled to be operated next. The information displayed in the side view SV is updated in real time according to the progress of the medical operations execution plan. The user can easily grasp the order of the medical devices operated by the user by viewing the side view SV. The side view SV may be displayed based on the operation information generated based on the situation information.

In the side view SV, the medical device 32 (a medical device B) which is currently being operated by the user, a medical device C to be operated next, and a medical device A to be operated next are displayed together with identification information. The identification information of the medical device is an icon indicating the medical device in FIG. 10, but is not limited thereto, and may be, for example, a photograph of the appearance of the medical device, a model name, a management number, or a combination thereof.

In the side view SV, a device screen or an external image of the medical device may be displayed in association with each medical device displayed in the operation order. In FIG. 10, the device screen (reduced version) is displayed for each medical device. The external image is, for example, a still image or a moving image captured by the camera provided in the room in which the medical device is installed.

The side view SV may display, for each of the plurality of medical devices A, B, and C, a remaining time of the process being executed or a time until the start of the process scheduled to be executed in the medical device. In FIG. 10, a timer is displayed in association with each of the medical devices A, B, and C. Timers T1 and T3 are drawn so as to draw a circle according to the lapse of time, and indicate the lapse of time of the process being executed. A timer T2 is drawn so as to erase circles as time passes, and indicates the time until the process starts. It is noted that the colors of the timers T1, T2, and T3 may be colors indicating the process type, and for example, may be matched with colors used to indicate the process type in the timeline TL.

The remaining time may be displayed numerically on each timer. In FIG. 10, the remaining time (the process ends in 3 minutes) of the process P22 is displayed numerically in the timer T1. In the timer T2, the time until the process P31 starts (the process starts in 1 minute and 20 seconds) is displayed numerically. In the timer T3, the remaining time of the process P13 (the process ends in 7 minutes and 30 seconds) is displayed numerically. It is noted that the elapsed time (the time elapsed from the start of the process) may be displayed for the process being executed instead of the remaining time.

It is noted that the side view SV may be displayed such that it is possible to grasp the type of the process being executed for each of the medical devices A, B, and C. For example, in the side view SV, a region where the device screen or the external image is displayed may be surrounded by a frame of a color corresponding to the process type, or the color of the timer may be the same as the color corresponding to the process type. In the example of FIG. 10, the color of a frame F1 and/or the timer T1 of the device screen of the medical device B is set to a color (for example, a red color) indicating scanning planning. In addition, the color of a frame F2 and/or the timer T2 of the device screen of the medical device C is set to a color (for example, a blue color) indicating patient care, and the color of the frame F3 and/or the timer T3 of the device screen of the medical device A is set to a color (for example, an orange color) indicating main scanning.

In addition, in FIG. 10, the medical devices are arranged in the operation order from the top to the bottom, but the mode of displaying the medical devices in the operation order is not limited thereto. For example, the display of the medical devices may be fixed, and numbers (1, 2, and 3) indicating the operation order may be displayed in association with identification information of the medical devices.

In addition, in FIG. 10, all the medical devices (in the present embodiment, three medical devices A, B, and C) provided in the medical information processing apparatus 1 are displayed in the side view SV, but the present invention is not limited thereto. For example, in a case where the number of medical devices is large, only a predetermined number of medical devices may be displayed in the side view SV in the operation order from a medical device which is currently being operated among the plurality of medical devices.

In addition, in the side view SV, only information on a medical device which is currently being operated by the user may be displayed, or only information on a medical device to be operated by the user next may be displayed.

Further, the side view SV is not limited to the case of being displayed in the side region of the display of the terminal 20, and may be displayed at any position.

In the operation view OV, the device screen of the medical device which is currently being operated by the user is displayed. In FIG. 10, the device screen of the medical device B which is currently being operated is displayed. The user operates the medical device while viewing the device screen displayed in the operation view OV. When the operation (process) of the medical device B ends, the device screen of the medical device C to be operated next by the user is displayed in the operation view OV.

In this manner, the information displayed in the operation view OV is updated in real time according to the progress of the medical operations execution plan. By viewing the operation view OV, the user can easily grasp the order of the medical devices operated by the user (which medical device should be operated now, which medical device should be operated next, and the like), the type of process for each medical device, the elapsed time of the process being executed, the time until the process starts, and the like.

In the execution status ES, progress information indicating the execution status of the workflow for each of the plurality of medical devices A, B, and C is displayed. In FIG. 10, the execution status ES is displayed in a table format. As illustrated in FIG. 12A (an enlarged view), the execution status of the workflow includes an inspection progress (progress information) indicating a process being executed by the medical device, an inspection interruption risk, and an inspection room status. In addition, biological information such as pulse and body temperature of the patient may be included in the execution status.

In the inspection progress, information (automatic/non-automatic information) indicating whether the process is automatically performed by the medical device, that is, whether the process is a process operated by the user may be displayed in addition to the name of the process being executed. In FIG. 12A, “automatic” indicates an automatic process that does not require a user operation.

In addition, in the execution status ES, interruption risk information indicating a risk that the process being executed is interrupted is displayed. The interruption risk information includes a possibility and a reason that the process being executed is interrupted. In FIG. 12A, for a patient P1, “patient position deviation” is displayed as the risk of interruption of patient positioning. In addition, for a patient P2, “contrast agent side effects” are displayed as the risk of interruption of main scanning. The state and condition of the patient such as the position deviation are determined by the server 10 (the processing circuit 15) from, for example, a positional relationship between the medical device and the patient photographed by the camera provided in the room in which the medical device is installed, and are displayed in the execution status ES.

Furthermore, in the example of FIG. 12A, for a patient P3, “deterioration in patient consciousness” is displayed as the risk of interruption of main scanning. This is determined by the server 10 (the processing circuit 15) based on the biological information such as the pulse and the body temperature of the patient, status of the inspection room grasped by the camera, and the like, and is displayed in the execution status ES.

In addition, in the execution status ES, inspection room status information indicating the status of the inspection room in which the medical device is installed is displayed. The inspection room status information is information indicating the status of the patient and the inspection room photographed by the camera (such as a real-time camera) of the inspection room. For example, information on the inspection room status, such as minute body movement of the patient and the technician responding to a sudden change of the patient, is displayed. The inspection room status information is an example of status information in the claims.

It is noted that, as illustrated in FIG. 12A, a display mode of the inspection interruption risk, the inspection room status, and the biological information may be changed depending on a content and a degree. For example, a cell may be displayed in yellow for a light to medium interruption risk, and a cell may be displayed in red for a significant interruption risk. In addition, a font or a color of a character indicating the interruption risk may be changed.

In this manner, the information displayed in the execution status ES is updated in real time according to the progress of the workflow, the state of the patient, the status of the inspection room, and the like. By viewing the execution status ES, the user can easily grasp the execution status of the workflow, the status of the site, and the state of the patient even in a case where the medical device is at a remote location. As a result, it can be determined whether a user or a local staff in charge needs to intervene.

It is noted that, as illustrated in FIG. 12B, information related to the execution status ES may be displayed in an image monitored by the camera in the room in which the medical device is installed. In FIG. 12B, a timer indicating a process being executed and an inspection interruption risk are displayed for each medical device together with the image of the camera. The timer is the same as that described in FIG. 10. By displaying the timer and the inspection interruption risk, the user can determine the necessity of intervention in the site. The screen in FIG. 12B may be displayed not as a part of the screen in FIG. 10 but by, for example, switching the screen in FIG. 10.

In addition, in the execution status ES, necessity of intervention of the user or the local staff in charge may be displayed.

In the process flow PF, the essential work and flow of the process which is currently being operated are displayed. FIG. 10 illustrates a display example in a case where scanning planning is being executed. In this display example, it is displayed that respective works of a tube current/a tube voltage, a reconstructed interval, an electrocardiogra synchronization setting, and an interlocking transfer setting should be sequentially performed. The display content of the process flow PF is updated by a change of the process being operated. By viewing the process flow PF, the user can easily grasp what should be performed in the process which is currently being operated, thereby making it possible to prevent omission of execution of the essential work.

It is noted that, in the process flow PF, the display modes may be different from each other, for example, colors may be changed so that the work being executed can be distinguished from other works. In FIG. 10, the “reconstructed interval” during the current work is displayed in a color different from the others.

As described above, in the screen 11, the timeline TL, the side view SV, the operation view OV, the execution status ES, and the process flow PF are arranged in one screen as the operation information. As a result, the user can easily and intuitively grasp the plurality of parallel workflows, the operation order of the user, the screen of the device which is currently being operated, the execution status of each workflow, the essential work and flow of the process which is currently being operated, and the like. In addition, by referring to the operation information, the user can efficiently perform the remote operation on the plurality of medical devices in parallel and give an appropriate instruction to the local staff in charge.

It is noted that it is not essential to display all of the timeline TL, the side view SV, the operation view OV, the execution status ES, and the process flow PF on one screen, and some of them may be displayed on the screen.

Next, a second screen example will be described with reference to FIG. 13. In this screen example, an operation view is displayed for each medical device. That is, as illustrated in FIG. 13, on a screen 12, operation views OV1, OV2, and OV3 of the medical devices are displayed in the operation order. More specifically, similarly to the side view SV of the screen 11, a medical device B displayed at the top is a medical device which is currently being operated, a medical device C displayed below the medical device B is a medical device scheduled to be operated next, and a medical device A displayed below the medical device C is a medical device schedule to be operated next. In the screen 12, each operation view includes identification information (here, an icon) of the medical device, a timer of the process, and a device screen.

It is noted that, in the operation views (in FIG. 13, the operation views OV2 and OV3 are illustrated) of the medical devices other than the medical device currently being operated, an external image may be displayed instead of the device screen.

In addition, the operation views OV1, OV2, and OV3 may be displayed such that it is possible to grasp the type of the process being executed. For example, a region in which the operation views OV1 to OV3 are displayed may be surrounded by a frame of a color corresponding to the process type, or a color of the timer may be the same as the color corresponding to the process type. In FIG. 13, a color of a frame F1 of the operation view OV1 and/or a timer T1 is a color (for example, a red color) indicating scanning planning. In addition, a color of a frame F2 and/or a timer T2 of the device screen of the operation view OV2 is set to a color (for example, a blue color) indicating patient care, and a color of a frame F3 and/or a timer T3 of the device screen of the operation view OV3 is set to a color (for example, an orange color) indicating main scanning.

In addition, in the screen 12, the operation views are arranged from the upper side to the lower side of the screen in the operation order of the medical device, but the present invention is not limited thereto. For example, a number indicating the operation order may be displayed in association with each operation view, or the operation order may be known by a display mode (a color, an effect, and the like) of the frames F1 to F3 of the operation view. For example, the frame of the operation view of the medical device to be currently operated may be set to a red color.

In addition, in FIG. 13, information corresponding to the number of medical devices (three in the present embodiment) included in the medical information processing apparatus 1 is displayed. The present invention is not limited thereto, and the side views may be displayed by a predetermined number of medical devices in the operation order from a medical device which is currently being operated.

It is noted that a display (a display unit) on which the above-described screen is displayed is not limited to the display of the terminal 20. The display may be a display (such as a stand tablet) provided in the medical devices 31, 32, and 33, a portable information terminal (such as a smartphone of the user and/or the local staff), and/or a display (a shared monitor) in a room in which the medical device is installed. By sharing the screen of the terminal 20 with the local staff in charge, it is possible to smoothly execute the workflow and to facilitate communication between the user and the local staff in charge when trouble occurs. Furthermore, the local staff in charge can provide the user with on-site information, consultation, advice, and the like as necessary.

A message (“please move a patient”, “monitoring of an inspection room is being performed”, and the like) from the user may be displayed on the displays provided in the medical devices 31, 32, and 33. The message may be made by voice via a speaker or the like of the medical device. This allows the local staff in charge to easily grasp what needs to be done at the present time point and the current status.

As explained above, the medical information processing apparatus according to the present embodiment acquires status information indicating a status of the plurality of medical devices and/or a status around the plurality of medical devices, generates operation information based on the acquired status information, and displays the generated operation information on the display unit. This allows, for example, a user such as a technician to remotely operate a plurality of medical devices efficiently and appropriately. It can also support appropriate communication between the user and the local staff.

Furthermore, in the present embodiment, the medical operations execution plan for executing a plurality of workflows in parallel by the plurality of medical devices is generated, and the medical device (that is, the medical device, which is an operation target of the user) to be operated by the user and the process executed by the medical device are displayed based on the medical operations execution plan. As a result, the user such as the technician can easily grasp the medical device to be operated and the process executed by the medical device. As a result, it is possible for the user to perform medical operations by the plurality of medical devices in parallel.

In the above embodiment, the medical information processing apparatus 1 is configured as a system including the server 10 and the terminal 20, but it is not limited to this. The medical information processing apparatus may be configured as a single device that includes the functions of the server 10 in the terminal 20.

It is noted that the term “processor” used in the above description means, for example, a central processing unit (CPU), a graphics processing unit (GPU), or circuits such as an application specific integrated circuit (ASIC) or a programmable logic device (for example, a simple programmable logic device (SPLD), a complex programmable logic device (CPLD), and a field programmable gate array (FPGA)). The processor implements a function by reading and executing a program stored in the memories 11 and 21. It is noted that, instead of storing the program in the memory 11, the program may be directly incorporated in the circuit of the processor. In this case, the processor implements the function by reading and executing the program incorporated in the circuit. It is noted that the processor is not limited to a case of being configured as a single processor circuit, and a plurality of independent circuits may be combined to be configured as one processor to implement the function. Furthermore, a plurality of components in FIGS. 2 and 4 may be integrated into one processor to implement the function.

Although several embodiments have been described above, these embodiments have been presented only as examples, and are not intended to limit the scope of the invention. The novel devices and methods described herein can be implemented in a variety of other forms. In addition, various omissions, substitutions, and changes can be made to the forms of the device and the method described in the present specification without departing from the gist of the invention. The appended claims and equivalents thereto are intended to include such forms and modifications as fall within the scope and spirit of the invention.

(Supplementary Note 1)

A medical information processing apparatus including:
a status information acquisition unit configured to acquire status information indicating a status of a plurality of medical devices connected to each other via a network and/or a status around the plurality of medical devices;
an operation information generation unit configured to generate, based on the status information, operation information indicating an operation to be performed on the medical device; and
a display control unit configured to control a display unit so as to display the generated operation information.

(Supplementary Note 2)

The medical information processing apparatus according to Supplementary note 1, in which:
the operation information generation unit is configured to generate, when there are a plurality of the operations to be performed on the plurality of medical devices, a plurality of pieces of the operation information based on the status information, in which each of the pieces of the operation information is generated for a corresponding one of the operations; and
the display control unit is configured to control the display unit so as to display the plurality of pieces of operation information side by side on a time axis.

(Supplementary Note 3)

The medical information processing apparatus according to Supplementary note 2, in which the display control unit is configured to control the display unit so as to display the plurality of pieces of operation information side by side in a timeline.

(Supplementary Note 4)

The medical information processing apparatus according to Supplementary note 3, in which the display control unit is configured to control the display unit so as to display, on the timeline, information for identifying a medical process being executed.

(Supplementary Note 5)

The medical information processing apparatus according to Supplementary note 1 or 2, further including a workflow information acquisition unit configured to acquire workflow information indicating a plurality of medical processes executed in parallel in the plurality of medical devices,
in which the operation information generation unit is configured to generate, based on the workflow information and the status information, the operation information.

(Supplementary Note 6)

The medical information processing apparatus according to Supplementary note 5, in which the respective medical processes of a plurality of workflows executed in parallel by the plurality of medical devices have different display modes depending on a type of the medical process and/or a status of the medical process.

(Supplementary Note 7)

The medical information processing apparatus according to Supplementary note 1, in which the display control unit is configured to display, on the display unit, the plurality of medical devices in an operation order.

(Supplementary Note 8)

The medical information processing apparatus according to Supplementary note 1, in which a type of a medical process being executed is displayed so as to be recognizable for each of the plurality of medical devices.

(Supplementary Note 9)

The medical information processing apparatus according to Supplementary note 1, in which the display control unit is configured to cause the display unit to display, for each of the plurality of medical devices, a remaining time or an elapsed time of a process being executed or a time until a start of a process scheduled to be executed by the medical device.

(Supplementary Note 10)

The medical information processing apparatus according to Supplementary note 1, in which the display control unit is configured to display, on the display unit, a device screen or an external image of each of the medical devices in association with each of the medical devices displayed in an operation order.

(Supplementary Note 11)

The medical information processing apparatus according to Supplementary note 1, in which the display control unit is configured to display, on the display unit, a device screen of a medical device currently being operated among the plurality of medical devices.

(Supplementary Note 12)

The medical information processing apparatus according to Supplementary note 1, in which the display control unit is configured to display, on the display unit, a device screen or an external image of each medical device in association with the plurality of medical devices.

(Supplementary Note 13)

The medical information processing apparatus according to Supplementary note 1, in which the display control unit is configured to display, on the display unit, an execution status of a workflow for each of the plurality of medical devices.

(Supplementary Note 14)

The medical information processing apparatus according to Supplementary note 13, in which the execution status includes progress information indicating a process being executed by the plurality of medical devices.

(Supplementary Note 15)

The medical information processing apparatus according to Supplementary note 14, in which the progress information includes information indicating whether a medical process being executed is automatically performed by the medical device.

(Supplementary Note 16)

The medical information processing apparatus according to Supplementary note 13, in which the execution status includes interruption risk information indicating a risk of interruption of a medical process being executed.

(Supplementary Note 17)

The medical information processing apparatus according to Supplementary note 13, in which the execution status includes inspection room status information indicating a status of an inspection room having the medical device installed therein.

(Supplementary Note 18)

The medical information processing apparatus according to Supplementary note 13, in which the execution status includes biological information of a patient.

(Supplementary Note 19)

The medical information processing apparatus according to Supplementary note 1, in which the display control unit is configured to display, on the display unit, an essential work and a flow in a process of the medical device currently being operated.

(Supplementary Note 20)

The medical information processing apparatus according to Supplementary note 1, in which the display unit is a display provided in at least one of a terminal for operating the plurality of medical devices, a portable information terminal of an operator who operates the plurality of medical devices, the medical device, or a room having the medical device installed therein.

(Supplementary Note 21)

The medical information processing apparatus according to Supplementary note 5, further including a medical operations execution plan generation unit configured to generate a medical operations execution plan, in which the medical operations execution plan is generated based on a timing at which a process of a certain medical device is allowed to transition to a process of another medical device, in which the timing is specified for each of a plurality of workflows executed in parallel by the plurality of medical devices.

(Supplementary Note 22)

The medical information processing apparatus according to Supplementary note 21, in which the medical operations execution plan generation unit includes:
a timing specification unit configured to specify the timing for each of the plurality of workflows;
a time calculation unit configured to calculate a process execution time of each process included in the plurality of workflows and a grace time between the processes; and
an operation order determination unit configured to determine, based on the timing, the process execution time, and the grace time, an operation order to sequentially operate the plurality of medical devices.

(Supplementary Note 23)

The medical information processing apparatus according to Supplementary note 22, in which the time calculation unit is configured to calculate the process execution time and the grace time based on at least one of performance information of the medical device, patient individual information, characteristic information of an operator who operates the plurality of medical devices, and a protocol.

(Supplementary Note 24)

The medical information processing apparatus according to Supplementary note 21, in which the medical operations execution plan generation unit is configured to generate the medical operations execution plan so as to prevent processes having a high risk of interruption from being continuously executed.

(Supplementary Note 25)

The medical information processing apparatus according to Supplementary note 21, in which the medical operations execution plan generation unit is configured to re-generate, when an unscheduled process that is not included in the medical operations execution plan is executed or when the process included in the medical operations execution plan is completed later or earlier than scheduled, the medical operations execution plan.

(Supplementary Note 26)

The medical information processing apparatus according to Supplementary note 21, in which the medical operations execution plan generation unit is configured to re-generate, when an abnormal finding is found by an inspection by the medical device and an additional inspection is approved, the medical operations execution plan.

(Supplementary Note 27)

The medical information processing apparatus according to Supplementary note 21, in which the medical operations execution plan generation unit is configured to re-generate, when a process delay is detected, the medical operations execution plan.

(Supplementary Note 28)

A medical information processing method including:
acquiring status information indicating a status of a plurality of medical devices connected to each other via a network and/or a status around the plurality of medical devices;
generating, based on the status information, operation information indicating an operation to be performed on the medical device; and
controlling a display unit so as to display the generated operation information.

1. A medical information processing apparatus comprising:
processing circuitry configured to
acquire status information indicating a status of a plurality of medical devices connected to each other via a network and/or a status around the plurality of medical devices;
generate, based on the status information, operation information indicating an operation to be performed on the medical device; and
control a display unit so as to display the generated operation information.

2. The medical information processing apparatus of claim 1, wherein the processing circuitry is further configured to:
generate, when there are a plurality of the operations to be performed on the plurality of medical devices, a plurality of pieces of the operation information based on the status information, in which each of the pieces of the operation information is generated for a corresponding one of the operations; and
control the display unit so as to display the plurality of pieces of operation information side by side on a time axis.

3. The medical information processing apparatus of claim 2, wherein the processing circuitry is further configured to control the display unit so as to display the plurality of pieces of operation information side by side in a timeline.

4. The medical information processing apparatus of claim 3, wherein the processing circuitry is further configured to control the display unit so as to display, on the timeline, information for identifying a medical process being executed.

5. The medical information processing apparatus of claim 1, wherein the processing circuitry is further configured to:
acquire workflow information indicating a plurality of medical processes executed in parallel in the plurality of medical devices; and
generate, based on the workflow information and the status information, the operation information.

6. The medical information processing apparatus of claim 5, wherein the respective medical processes of a plurality of workflows executed in parallel by the plurality of medical devices have different display modes depending on a type of the medical process and/or a status of the medical process.

7. The medical information processing apparatus of claim 1, wherein the processing circuitry is further configured to cause the display unit to display, for each of the plurality of medical devices, a remaining time or an elapsed time of a process being executed or a time until a start of a process scheduled to be executed by the medical device.

8. The medical information processing apparatus of claim 1, wherein the processing circuitry is further configured to display, on the display unit, a device screen or an external image of each of the medical devices in association with each of the medical devices displayed in an operation order.

9. The medical information processing apparatus of claim 1, wherein the processing circuitry is further configured to display, on the display unit, a device screen of a medical device currently being operated among the plurality of medical devices.

10. The medical information processing apparatus of claim 1, wherein the processing circuitry is further configured to display, on the display unit, an execution status of a workflow for each of the plurality of medical devices.

11. The medical information processing apparatus of claim 10, wherein the execution status includes progress information indicating a process being executed by the plurality of medical devices.

12. The medical information processing apparatus of claim 10, wherein the execution status includes interruption risk information indicating a risk of interruption of a medical process being executed.

13. The medical information processing apparatus of claim 10, wherein the execution status includes biological information of a patient.

14. The medical information processing apparatus of claim 1, wherein the display unit is a display provided in at least one of a terminal for operating the plurality of medical devices, a portable information terminal of an operator who operates the plurality of medical devices, the medical device, or a room having the medical device installed therein.

15. A medical information processing method comprising:
acquiring status information indicating a status of a plurality of medical devices connected to each other via a network and/or a status around the plurality of medical devices;
generating, based on the status information, operation information indicating an operation to be performed on the medical device; and
controlling a display unit so as to display the generated operation information.
